(19) United States
(12) Patent Application Publication
(10) Pub. No.: US 2009/0028960 A1
(43) Pub. Date: Jan. 29, 2009

(54) CALCIUM PHOSPHATE-BASED ADHESIVE FORMULATION FOR BONE FILLING

(76) Inventors: Alain Leonard, Caixon (FR); Stephane Goncalves, Balma (FR); Juliette Fitremann, Toulouse (FR); Ariane Bercier, Creteil (FR)

Correspondence Address: MINTZ LEVIN COHN FERRIS GLOVSKY & POPEO, ONE FINANCIAL CENTER, BOSTON, MA, 02111, US

(21) Appl. No.: 11/830,019

(22) Filed: Jul. 30, 2007

(30) Foreign Application Priority Data
Jul. 25, 2007 (FR) 07 05405

(51) Int. Cl. A61K 33/42; A61P 19/00

(52) U.S. Cl. 424/602

(57) ABSTRACT

The present invention relates to a calcium phosphate-based formulation for bone filling, comprising at least one adjuvant giving adhesion properties, said at least one adjuvant can be selected from the saccharide head surfactant group.

The bone cement described in the present invention is characterised by adhesive properties particularly advantageous facilitating the setting of the cement and offering a better containment of the bone-cement or bone-cement-prosthesis interface.

### RELATED APPLICATION

The present application claims the benefit of priority of French Patent Application FR 07 05405, filed on Jul. 25, 2007 and incorporated by reference, herein, in its entirety.

The present invention relates to biomaterials useful in the orthopaedic and dental sector. More particularly, it relates to materials enabling filling of bone or dental defects, and the colonization of such materials with live cells of the tissue wherein they are to be implanted.

Biomaterials are presently developed in order to propose, in surgical practice, various types of materials used to substitute damaged tissues and/or promote and accelerate their healing and natural regeneration.

In the orthopaedic and dental field, more specifically, materials with a very similar chemical structure to the mineral phase of bone tissue or teeth have been developed. Numerous bone substitutes already currently exist on the market. They exist in various forms and/or applications. Indeed, calcium phosphate ceramics are found that have been used for over twenty years as pellets, powder, and other geometric forms which can thus fit the defects to be filled. A new route has been made available for ten years of so, which makes use of cements or pastes, which are injectable and therefore enable “mini-invasive” surgery which is less traumatic for the patient. Such cements are prepared using a mixture of a calcium phosphate solid phase and an aqueous solution containing calcium phosphate salts as well, and optionally various additives (e.g., Lacout et al., FR 2 776 282, and Lacout et al., FR 2 805 748). Subsequent to the acid-base reactions that occur during the mixing of the powder and the liquids, various steps are observed. A first step is the setting of the cement, following which the cement remains stable in terms of shape. A second step is the hardening phase, during which the hardness of the material increases over time. The hardening progressively gives the material a higher mechanical resistance, compatible with the supporting functions of the skeleton. This setting phenomenon offers several major advantages: first, the initial paste form enables the material to conform to all sorts of defective bone cavities. Then, the formulation of these cements, close to the hydroxyapatite of the bone tissue, makes them both biocompatible and osteoconductive. Finally, the microporosity of the phosphocalcium cements enables the circulation of biological fluids, thus facilitating colonization by bone cells and progressive bioresorption. At the present time, several phosphocalcium have been developed and marketed. These materials may also be considered as substrates or matrices for tissue engineering.

To date, the bone-cement or bone-cement-prosthesis interface has been the subject of extensive studies, which have demonstrated its behaviour and its properties during the positioning of the cement. In the meantime, the manner in which the paste physically makes contact with the bone or with the prostheses, during the application thereof, has never been studied in terms of physical adherence. Yet, during the application of the substitution material, said material is assumed to fill the unoccupied spaces, either of the bone itself, in the event of a fracture, or osteoporosis for example, or between a prosthesis and the bone, including on the interfaces between materials. If the unoccupied spaces are filled poorly, they may be the site of anarchic cell proliferation, unfavourable for healing. Therefore, the mechanisms that occur at the bone-filling material interface are of primary importance in cell repair mechanisms. The use of a cement tending to adhere both to the bone and, if applicable, to a prosthesis positioned on the same site, will enable the application of a bone-material interface of improved quality, improved filling and easier application, due to the adherence and cohesion of the paste. In addition, the adhesion properties will make it possible to reduce significantly any migration reaction once fitted in the body. These properties may also allow a more precise positioning of bone fragments resulting from a multiple fracture within the adhesive cement paste.

The formulation of adhesive phosphocalcium cement pastes, with the aim of improving the cement properties such as described above, has never been dealt with before. The adhesive properties or “tackiness” are generally provided by the positioning of a polymer between the surfaces of two materials (Handbook of Adhesive Technology, A. Pizzi, K. L. Mittal, Marcel Dekker Ed., 2003). It may consist in particular of natural polymers, such as proteins, polysaccharides or it may consist of synthetic polymers. These molecules are capable of developing numerous weak interactions with different types of surfaces. In the case of polysaccharides for example, they will form numerous hydrogen bonds with polar surfaces, through saccharide groups. Another parameter involved in adhesion phenomena is the viscoelasticity of the compound used.

Surfactants derived from sugars, molecules consisting of a saccharide hydrophilic part and a hydrophobic part (hydrocarbon chain), combine the ability to form strong hydrogen bonds with polar substrates, and hydrophobic interactions with apolar substrates. Another characteristic of these molecules is their ability to form lyotropic phases in the presence of water, i.e. organized phases. These solutions, according to their concentration, may display gel behaviors, or, in the broadest sense, viscoelastic properties of interest (J. Am. Oil Chemists Soc., 1992, 69, (7), 660-666). However, generally, surfactants derived from sugars are used for their surfactant properties which facilitate the stabilization of emulsions, foams and solid-liquid dispersions. They usually are non-toxic molecules, and for this reason, they are used more specifically in cosmetics, food processing, or in detergents. They are also specifically used for lubrication, i.e. to obtain a non-stick effect during mould release, in the fields of food processing or pharmaceutical formulation (see for example the technical manuals for sucrose esters produced by Stéarinerie Dubois or Mitsubishi-Kagaku Foods Corporation).

In this way, in the case of phosphocalcium cements, surfactants from different families (anionic, cationic, zwitterionic, various non-ionic families, including some saccharide head surfactants) have been added either to facilitate the miscibility of a hydrophobic liquid in the powder-aqueous solution mixture (Bohner, U.S. Pat. No. 6,642,285), or to generate porosity by means of air bubble carry-over (Patent WO 2004/000374 and WO 2005/084726). In this case, surface tension lowering properties induced by these molecules have been used. Moreover, Ginebra at al. (Patent WO 2006/030054) also propose the addition of surfactants in phosphocalcium cements and, among other things, saccharide head surfactants which are added, on the one hand, to generate porosity, by formulating a solid cement foam. On the other hand, they are added to improve injectability. In the latter case, by adsorbing on the surface of the cement particles, they improve the solid/liquid suspension, and act as lubricants by improving the mutual sliding of the particles. However, such formulations are of a limited interest, particularly in terms of adhesion.

Indeed, the occurrence of adhesion reactions by means of the addition of such molecules into a phosphocalcium mixture has never been described. This observation represents the starting point of the present invention. Unexpectedly, it was thus found that the addition of sugar-derived surfactants, alkylpolyglucosides, alkylpolyglycosides, sucrose fatty acids, sucroglycerides, sorbitan esters, made it possible to enhance strongly the adhesive capabilities of the paste, or, in other words, the adhesion properties of the cement paste with respect to various substrates, such as bone, metals, synthetic or natural polymers. More specifically, a hydrophile—lipophile balance (or “HLB”, corresponding to the ratio of the polar moiety of the surfactant with respect to the apolar moiety) made it possible to obtain a high degree of adhesion. Nevertheless, the addition of these molecules allows to retain the setting and hardening characteristics of the cements. On the other hand, the addition of non-surfactant polysaccharides or monosaccharides, disaccharides or oligosaccharides did not make it possible to observe such a phenomenon.

The present invention related to a calcium phosphate-based formulation for bone filling wherein at least one adjuvant has been added allowing to increase the adhesion of the paste significantly, before setting, with respect to various substrates such as bone, metals, synthetic or natural polymers. The satisfactory adherence of the paste on the operating site will enable the formation of an improved bone-filling material interface, and very easy application.

In this invention, the term “adhesion” defines the property displayed by the cement paste to adhere to a substrate, as characterized and measured by the adherence test described in the present application. The measurements carried out provide two quantitative values for this property, the adhesion strength and the adhesion energy. This property may also be referred to as “tackiness” (immediate adhesion in contact with substrate). The terms “adhesive” (in the common sense) or “adherence” (tests characterizing such adhesion) may also be used to refer to the property observed.

The innovative aspect of the invention firstly relates to the incorporation of saccharide head surfactants with the cement powder, with a view to giving the cement paste adhesive properties. Without being able to explain exhaustively all the reactions involved, it would seem that these adjuvants, due to their hydrophilic properties (provided by the sugar head), their viscoelastic properties (provided by the self-organization ability of these amphiphilic molecules), and the ability of these molecules to be adsorbed onto various substrates via weak bonds (hydrogen bonds of sugar and hydrophobic bonds of fatty chains), generate an adhesion phenomenon of the paste on various substrates, bone, metal, synthetic polymer, or natural polymer.

The adjuvants are preferentially: sorbitan esters, sucrose fatty esters (sucrose laurate, sucrose myristate, sucrose palmitate, sucrose stearate, sucrose oleate, sucrose behenate, sucrose erucate, pure or in mixtures of mono, di, tri, tetrasubstitutes and more), sucroglycerides, alkylpolyglucosides (with glucose polar head, and with octyl, decyl, dodecyl, tetradecyl, hexadecyl, octadecyl alkyl chain), alkylpolyglycosides (with polar head consisting of any type of saccharide, and with octyl, decyl, dodecyl, tetradecyl, hexadecyl, octadecyl alkyl chain, pure or in mixtures). These adjuvants also display a good biocompatibility profile, which makes them suitable products for their use for in vivo implantation.

An adjuvant selected in a preferential manner to produce the formulation according to the invention is a sucrose fatty ester containing 50 to 100 weight % of monoester.

The formulation according to the invention may also be defined in that it comprises at least one adjuvant displaying a hydrophile-lipophile balance between 10 and 20.

The weight percentage of adjuvant in the final mixture of the formulation according to the invention is between 0.1 and 25, preferentially between 1 and 10, more preferentially between 7 and 10.

Moreover, the phosphocalcium cements also correspond to any type of mixture of calcium phosphate powders, with or without another adjuvant, which, mixed with an aqueous solution containing calcium phosphates and other adjuvants or not, result in a setting and hardening phenomenon as described above or in the French patent FR 2 776 282.

The final calcium to phosphate ratio of the formulation according to the invention is between 1.4 and 1.8, preferentially between 1.6 and 1.7.

The formulation obtained in this way according to the invention displays an adhesion energy up to 20 times greater than a conventional formulation prepared without adjuvant as defined in the present application.

In another aspect of the embodiment of the invention, saccharide head surfactants (in powder or liquid form) are mixed with the solid phase of the cement rather than in the liquid phase, which enables improved preservation of their properties during long-term storage. Both powders are mixed intimately by means of a mechanical method, either manually, or using an industrial powder mixer or grinder.

The sugar derivatives described above are added to the cement powder in variable proportions and the liquid phase is added in order to obtain the cement paste. The adhesion of this paste to various substrates was determined by adherence tests recorded using a mechanical testing machine (measurement of strength as function of displacement), as defined below and illustrated using the following figures:

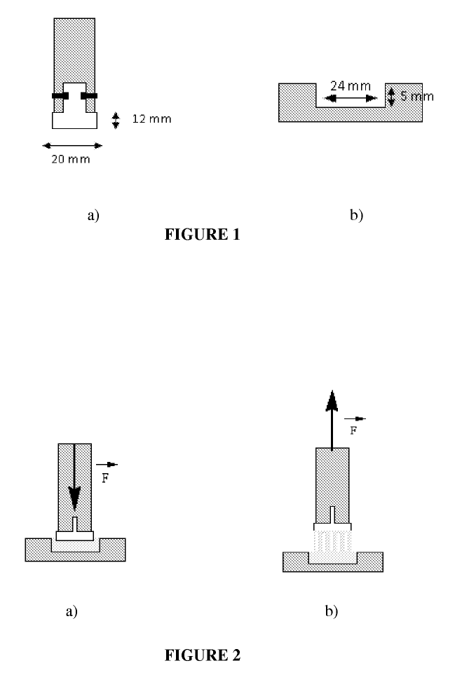

FIG. 1: mobile device used to perform adhesion tests, a) mobile head, b) trough.

FIG. 2: operation of mobile device during an adhesion test of a formulation according to the invention, a) compression of the formulation, b) traction of the formulation.

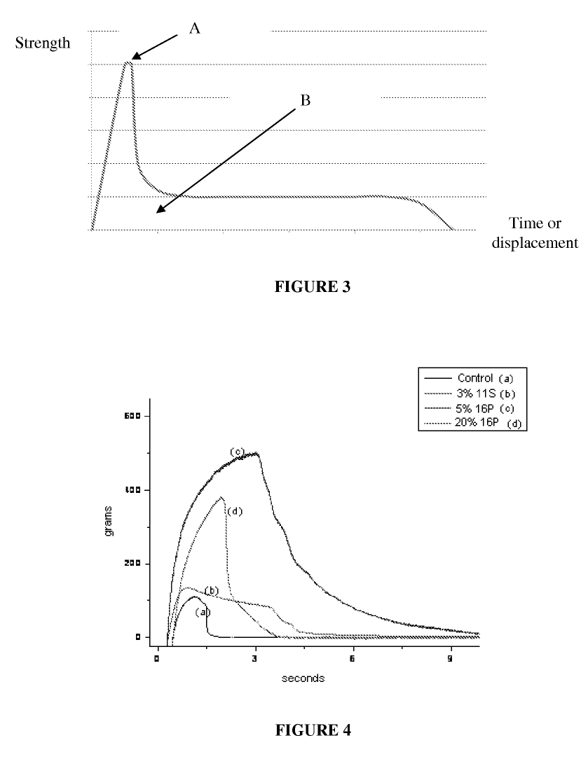

FIG. 3: standard adhesion curve with A corresponding to the adhesion strength and B corresponding to the adhesion Energy.

The device used for these tests comprises two parts (FIG. 1):

an aluminum plate wherein a flat-bottomed trough, 24 mm in diameter and 5 mm high, has been machined, secured on the base of the mechanical testing machine;

an aluminum piston secured on the arm of the mechanical testing machine and whereon machine flat “heads” in different materials can be fitted. The surface of these heads is polished, and has a diameter of 20 mm. The various materials used are: aluminium, steel, stainless steel, Plexiglas (polymethylmethacrylate), brass, nylon, Teflon and bone (bovine tibia).

The adherence test is performed in two steps (FIG. 2):

(i) the cement is mixed, introduced into the “trough” and levelled flush with the top edge. At mixing for 5 min 30, a force of 800 g is applied until the head of the mobile device is inserted into the paste by 2 mm.

(ii) the second part of the experiment consists of a traction test. The arm of the mobile assembly is raised at a constant speed (0.2 mm/second) until it returns to the initial point.

The value measured during this test is the necessary force to be applied to the arm of the mobile device, during the traction test, to maintain the speed at 0.2 mm/second. The resistance offered by the cement varies during the test, according to its adhesive properties. Curves such as (FIG. 3) are obtained.

On the basis of these curves, two characteristic values are determined: the adhesion strength (N/mm²) corresponding to the force peak of the curve and the adhesion energy (kJ/m²) which is proportional to the area under the curve.

The tests were performed comparatively between “control” cements, wherein the cement powder does not contain any surfactant, and cements comprising the different sugar-derived adjuvants mentioned in the present invention, introduced in different percentages by weight. The adhesion of the cement pastes with respect to different materials was also measured comparatively. The materials selected are as follows, including materials with which the cement is liable to come into contact during an orthopaedic surgery operation: aluminium, steel, stainless steel, Plexiglas (polymethylmethacrylate), brass, nylon, Teflon and bone (bovine tibia).

The results of these tests are represented by curves illustrating different adhesion levels according to the formulations produced according to the invention and the substrates used. These curves are shown in the following figures:

FIG. 4: adhesion curves of various formulations according to the invention on nylon substrate.

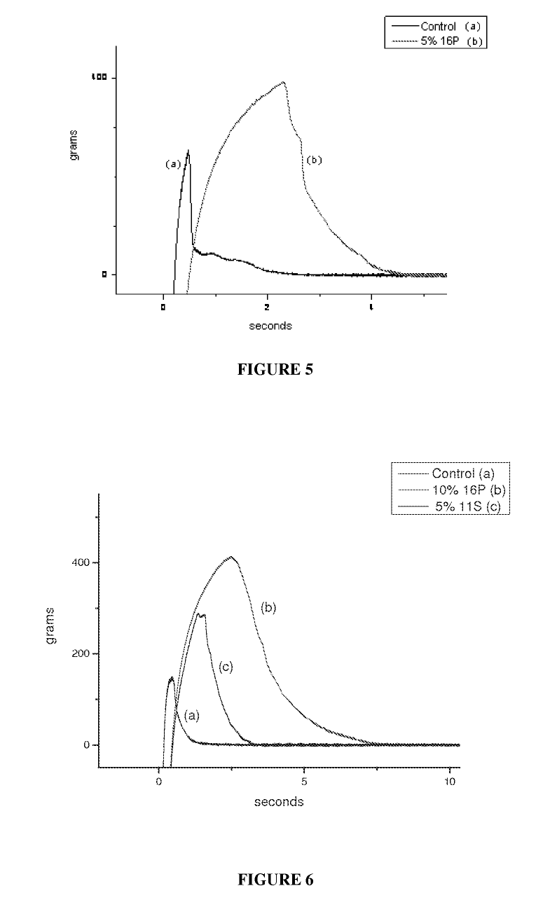

FIG. 5: adhesion curves of various formulations according to the invention on stainless steel substrate.

FIG. 6: adhesion curves of various formulations according to the invention on bone substrate.

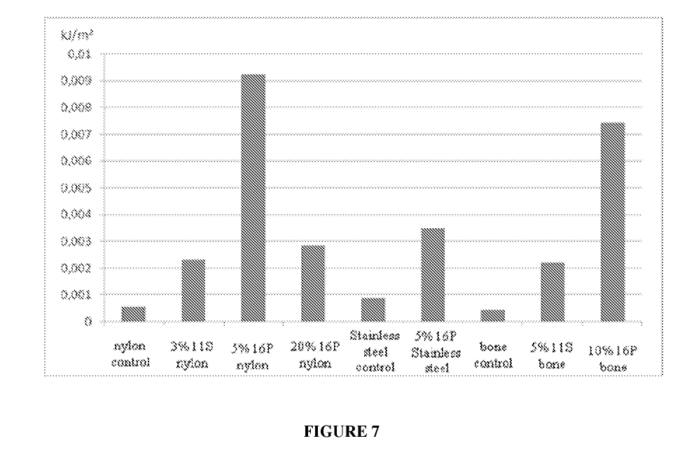

FIG. 7: adhesion energy corresponding to the different formulations tested in this way.

The measurements demonstrate that the adhesion properties of the paste, displayed by the adhesion strength and the adhesion energy, on a piston made of different materials (bone, steel, stainless steel, Plexiglas, nylon, aluminium), are increased significantly when sugar-derived surfactants are added as compared to the adjuvant-free cement. It was also observed that there is an optimal hydrophile-lipophile balance (HLB) of the surfactant, and an optimal percentage of adjuvant enabling optimum adhesion of the paste. In the case of sucrose fatty esters, the HLB is defined as being equal to 0.2 times the weight percentage of monosubstitute esters (monoesters) in the mixture (scale defined by the suppliers, see also A.-S. Muller et al., 2002). Moreover, the addition of non-amphiphilic polysaccharides or monosaccharides, disaccharides or oligosaccharides did not make it possible to observe such a phenomenon.

### EXAMPLES

The invention will be better understood with reference to the following examples, which are not limiting the scope thereof, however.

According to a preferred cement preparation method, a cement powder comprising a-TCP (a-tricalcium phosphate), TTCP (tetracalcium phosphate), and sodium glycerophosphate (Cementek®) or the same powder supplemented with polydimethylsiloxane (Cementek® LV) is mixed with sugar-derived surfactants, with a cement powder/surfactant powder ratio such that the weight percentage of the surfactant in the final powder+liquid mixture is between 0.1 and 25%. Subsequent to the mixing of powders, a phosphoric acid and calcium hydroxide solution (such that the final Ca/P ratio is equal to 1.634) (Teknimed) is added in a solid/liquid ratio of 0.43 ml/g. The resulting mixture is mixed for at least 3 minutes. The resulting paste readily takes on a tacky appearance. The adhesion energy is measured using a mechanical testing machine and it is compared to that of a surfactant-free control. The following examples illustrate more specifically the embodiment of the invention.

Formulation 1: 2.001 g of Cementek® (Teknimed) and 0.145 g of sucrose stearate with an HLB=11 (abbreviation: 11S)—i.e. 5% of 11S with respect to the total weight of the cement+liquid—were mixed for 2 minutes. To the mixture of powders, 0.63 g of a calcium phosphate acid solution (Teknimed) (at t=0) was added and the resulting mixture was mixed for 3 minutes. The Liquid/Solid ratio is 0.43. The resulting paste, which had a sticky texture, was then introduced into the aluminium trough and levelled flush with the top edge of same. At t=5′30″, the adhesion test was performed using a bone “head” piston. The adhesion energy measured was 2.2.10⁻³ kJ/m², i.e. 5.5 times greater than the adhesion energy of a surfactant-free control cement sample.

The same test was performed using 3% of 11S, and a nylon “head”. The adhesion energy measured was 2.3.10⁻³ kJ/m², i.e. 3.8 times greater than the adhesion energy of a surfactant-free control cement sample.

Formulation 2: 2.00 g of Cementek® (Teknimed) and 0.287 g of sucrose palmitate with an HLB=16 (abbreviation: 16P)—i.e. 10% of 16P with respect to the total weight of the cement+liquid—were mixed for 2 minutes. To the mixture of powders, 0.861 g of a calcium phosphate acid solution (Teknimed) (at t=0) was added and the resulting mixture was mixed for 3 minutes. The Liquid/Solid ratio was 0.43. The resulting paste, which had a sticky texture, was then introduced into the aluminium trough and levelled flush with the top edge of same. At t=5′30″, the adhesion test was performed using a bone “head” piston. The adhesion energy measured was 7.4.10⁻³ kJ/m², i.e. 18.5 times greater than the adhesion energy of a surfactant-free control cement sample.

The same test was performed using 5% of 16P, and a nylon “head”. The adhesion energy measured was 9.3.10⁻³ kJ/m², i.e. 15.5 times greater than the adhesion energy of a surfactant-free control cement sample.

The same test was performed using 5% of 16P, and a stainless steel “head”. The adhesion energy measured was 4.6.10⁻³ kJ/m², i.e. 7.1 times greater than the adhesion energy of a surfactant-free control cement sample.

The same test was performed using 20% of 16P, and a nylon “head”. The adhesion energy measured was 2.9.10⁻³ kJ/m², i.e. 4.8 times greater than the adhesion energy of a surfactant-free control cement sample.

Formulation 3: 1.9997 g of Cementek®(Teknimed) and 0.2891 g of sucrose palmitate with an HLB=5 (abbreviation: 5S)—i.e. 10% of 5S with respect to the total weight of the cement+liquid—were mixed for 2 minutes. To the mixture of powders, 0.862 g of a calcium phosphate acid solution (Teknimed) (at t=0) was added and the whole was mixed for 3 minutes. The Liquid/Solid ratio was 0.43. The resulting paste, which had a sticky texture, was then introduced into the aluminium trough and levelled flush with the top edge of same. At t=5′30″, the adhesion test was performed using a nylon “head” piston. The adhesion energy measured was 1.8.10⁻³ kJ/m², i.e. 3 times greater than the adhesion energy of a surfactant-free control cement sample.

Formulation 4: 2.0003 g of Cementek®(Teknimed) and 0.2838 g of sucrose palmitate with an HLB=16 (abbreviation: 16L)—i.e. 10% of 16L with respect to the total weight of the cement+liquid—were mixed for 2 minutes. To the mixture of powders, 0.861 g of a calcium phosphate acid solution (Teknimed) (at t=0) was added and the whole was mixed for 3 minutes. The Liquid/Solid ratio was 0.43. The resulting paste, which had a sticky texture, was then introduced into the aluminium trough and levelled flush with the top edge of same. At t=5′30″, the adhesion test was performed using a bone “head” piston. The adhesion energy measured was 4.6.10⁻³ kJ/m², i.e. 11.5 times greater than the adhesion energy of a surfactant-free control cement sample.

Formulation 5: 2.0008 g of Cementek®(Teknimed) and 0.145 g of a mixture of palmityl glucoside and palmitic alcohol (alkylpolyglucoside Montanov 68EC, Seppic)—i.e. 5% of 68EC with respect to the total weight of the cement+liquid—were mixed for 2 minutes. To the mixture of powders, 0.863 g of a calcium phosphate acid solution (Teknimed) (at t=0) was added and the mixture was mixed for 3 minutes. The Liquid/Solid ratio was 0.43. The resulting paste, which had a sticky texture, was then introduced into the aluminium trough and levelled flush with the top edge of same. At t=5′30″, the adhesion test was performed using a nylon “head” piston. The adhesion energy measured was 1.4.10⁻³ kJ/m², i.e. 2.3 times greater than the adhesion energy of a surfactant-free control cement sample.

The same test was performed using 5% of Montanov 68EC, and a bone “head”. The adhesion energy measured was 1.9.10⁻³ kJ/m², i.e. 4.8 times greater than the adhesion energy of a surfactant-free control cement sample.

The same test was performed using 5% of Montanov 14 (mixture of myristyl glucoside and myristic alcohol), and a bone “head”. The adhesion energy measured was 1.1.10⁻³ kJ/m², i.e. 2.8 times greater than the adhesion energy of a surfactant-free control cement sample.

Formulation 6: 2.0016 g of Cementek LVâ (Teknimed) and 0.1463 g of sucrose palmitate with an HLB=16—i.e. 5% of 16P with respect to the total weight of the cement+liquid—were mixed for 2 minutes. To the mixture of powders, 0.863 g of a calcium phosphate acid solution (Teknimed) (at t=0) was added and the whole was mixed for 3 minutes. The Liquid/Solid ratio was 0.43. The resulting paste, which had a sticky texture, was then introduced into the aluminium trough and levelled flush with the top edge of same. At t=5′30″, the adhesion test was performed using a nylon “head” piston. The adhesion energy measured was 3.8.10⁻³ kJ/m², i.e. 7.6 times greater than the adhesion energy of a surfactant-free control cement sample.

The same test was performed using 5% of 16P, and a stainless steel “head”. The adhesion energy measured was 3.5.10⁻³ kJ/m², i.e. 3.9 times greater than the adhesion energy of a surfactant-free control cement sample.

The same test was performed using 5% of Montanov 14, and a nylon “head”. The adhesion energy measured was 1.3.10⁻³ kJ/m², i.e. 2.6 times greater than the adhesion energy of a surfactant-free control cement sample.

The same test was performed using 5% of 16L, and a bone “head”. The adhesion energy measured was 1.8.10⁻³ kJ/m², i.e. 4.5 times greater than the adhesion energy of a surfactant-free control cement sample.

1. Calcium phosphate-based formulation for bone filling, characterised in that it comprises at least one adjuvant giving adhesion properties.

2. Formulation according to claim 1, wherein said at least one adjuvant is selected from the saccharide head surfactant group.

3. Formulation according to claim 2, wherein the saccharide head surfactants are selected from the group of sorbitan esters, sucrose fatty esters, sucroglycerides, alkylpolyglucosides and alkylpolyglycosides.

4. Formulation according to claim 3, wherein the saccharide head surfactants are preferentially sucrose fatty esters containing 50 to 100% by weight of monoester.

5. Formulation according to claim 1, wherein said at least one adjuvant has an HLB between 10 and 20.

6. Formulation according to claim 1, comprising 0.1 to 25 weight % of adjuvant in the final mixture.

7. Formulation according to claim 6, comprising 1 to 10 weight % of adjuvant in the final mixture.

8. Formulation according to claim 7, comprising 7 to 10 weight % of adjuvant in the final mixture.

9. Formulation according to claim 1, wherein the final Ca/P ratio is between 1.4 and 1.8.

10. Formulation according to claim 9, wherein the final Ca/P ratio is preferentially between 1.6 and 1.7.

11. Formulation according to claim 1, wherein the formulation adhesion energy is enhanced by a coefficient of up to 20 times the value of an adjuvant-free mixture.

12. Formulation according to claim 1, wherein the adjuvant is added to a calcium phosphate powder mixture and to the solid bone cement phase.
